(19) United States
(12) Patent Application Publication
(10) Pub. No.: US 2018/0368706 A1
(43) Pub. Date: Dec. 27, 2018

(54) METHOD AND A SYSTEM TO MEASURE BLOOD PRESSURE WITH AUTOMATIC HEART REFERENCE PRESSURE COMPENSATION

(71) Applicant: Edwards Lifesciences Corporation, Irvine, CA (US)

(72) Inventors: Feras Al Hatib, Irvine, CA (US); Mark A. Konno, Laguna Beach, CA (US); Luong N. Phan, San Clemente, CA (US); Lindon A. Baker, Yorba Linda, CA (US); Richard A. Gros, Anaheim Hills, CA (US); Aaron R. London, Los Alamitos, CA (US)

(21) Appl. No.: 15/776,750

(22) PCT Filed: Nov. 18, 2015
(86) PCT No.: PCT/US2015/061259

§ 371 Date: May 16, 2018

(51) Int. Cl. A61B 5/0215; A61B 5/03

(52) U.S. Cl. CPC A61B 5/02156; A61B 5/031; A61M 2025/0003; A61B 5/02158; A61B 5/02152

(57) ABSTRACT

Disclosed is an apparatus, system, and method for compensating for hydrostatic pressure offset in transducer-based pressure measurements. The system may comprise: a measurement pressure transducer to measure an apparent fluid pressure at a measurement site, a reference pressure transducer to measure a hydrostatic pressure caused by a level difference between the measurement pressure transducer and the measurement site, and a controller to generate a corrected fluid pressure measurement based on the apparent fluid pressure and the hydrostatic pressure, wherein the measurement pressure transducer and the reference pressure transducer are placed at a same first level, and the measurement site and an end of a fluid-filled tube connected to the reference pressure transducer are at a same second level.

### CROSS-REFERENCE TO RELATED APPLICATIONS

This application claims the benefit of International Patent Application No. PCT/US2015/61259, filed Nov. 18, 2015, the contents of which are incorporated by reference herein in their entireties for all purposes.

### BACKGROUND

### Field

The present invention relates to pressure transducers. In particular, embodiments of the invention relate to a method and apparatus for automatic compensation of hydrostatic pressure in transducer pressure measurements without manual leveling or zeroing.

### Relevant Background

Disposable pressure transducers (DPTs) are widely used to monitor blood pressure signals in a patient's vein or artery. DPTs can also be used to monitor intracranial pressure as well as a wide variety of other types of pressure measurements. The pressure signals generated by DPTs may be used for a number of monitoring and diagnostic applications, and are often connected to a patient monitor to display a graphical depiction of pressure versus time. Other patient monitors may employ sophisticated algorithms to derive volumetric and hemodynamic parameters from the pressure signal.

A DPT is typically mounted near the patient and connected to the patient's vein, artery, cranium, or other part of the body via a catheter and a fluid-filled tube. The pressure signal is transmitted from the measurement site (e.g., vein, artery, or cranium) via the catheter and the fluid-filled tube, to the pressure transducer. The pressure transducer is typically located in a plastic enclosure that ensures connectivity to the fluid-filled catheter-tubing system on one side and the patient monitor on the other side. The term DPT usually refers to a system that includes the enclosure hosting the pressure transducer, the transducer itself, and the respective connectors.

To obtain accurate pressure measurements, the DPT must be mounted level with the measurement site. For example, for arterial pressure measurement, the DPT should be mounted at the same level as the patient's heart or the phlebostatic axis. If the DPT is above or below the patient's heart level, a hydrostatic pressure offset occurs due to the height of the fluid column in the tubing, and the apparent pressure signal generated by the DPT is no longer accurately representative of the blood pressure.

The presence of a hydrostatic pressure may introduce significant errors in the pressure measurement. A known method to reduce the effect of the hydrostatic pressure is through manual zeroing of the transducer with respect to atmospheric pressure. The leveling is usually performed by visual estimation or by using a carpenter's level or a laser leveler and includes a number of manual procedures that may be time consuming in many clinical conditions. Moreover, the manual zero referencing is proven to be unreliable with significant inter-user variability. Additionally, the hydrostatic pressure could have a significant impact on the pressure measurement when the patient moves, or when the patient bed is moved or inclined, or when the patient needs to be rotated in a continuous fashion. In these types of clinical conditions, manual zeroing of the pressure transducer could be a challenge.

### SUMMARY

Embodiments of the invention may relate to an apparatus, system, and method for reliable, continuous, automatic, and real-time compensation of hydrostatic pressure offset without the need of manual zeroing and leveling. The system may comprise: a measurement pressure transducer to measure an apparent fluid pressure at a measurement site, a reference pressure transducer to measure a hydrostatic pressure caused by a level difference between the measurement pressure transducer and the measurement site, and a controller to generate a corrected fluid pressure measurement based on the apparent fluid pressure and the hydrostatic pressure, wherein the measurement pressure transducer and the reference pressure transducer are placed at a same first level, and the measurement site and an end of a fluid-filled tube connected to the reference pressure transducer are at a same second level.

### BRIEF DESCRIPTION OF THE DRAWINGS

FIG. 1 is a diagram illustrating an example disposable pressure transducer (DPT), with which embodiments of the invention may be practiced.

FIG. 2 is a diagram illustrating an example environment in which embodiments of the invention may be practiced.

FIG. 3 is a diagram illustrating an example heart reference system (HRS), according to one embodiment of the invention.

FIG. 4 is a block diagram illustrating an example hydrostatic pressure compensation system comprising electronic modules for reliable, continuous, automatic, and real-time compensation of hydrostatic pressure, according to one embodiment of the invention.

FIG. 5 is a diagram illustrating circuit components of an example DPT converter, according to one embodiment of the invention

FIG. 6 is a diagram illustrating an example DPT plate comprising various electronic modules.

FIG. 7 illustrates a front view and a back view of an example DPT plate.

### DETAILED DESCRIPTION

Embodiments of the invention generally relate to an apparatus, system, and method for reliable, continuous, automatic, and real-time compensation of hydrostatic pressure offset without the need of manual zeroing and/or leveling of a DPT. This provides automatic and real-time compensation for static pressure variations caused by changes in relative height between a measurement site and the DPT mounting location.

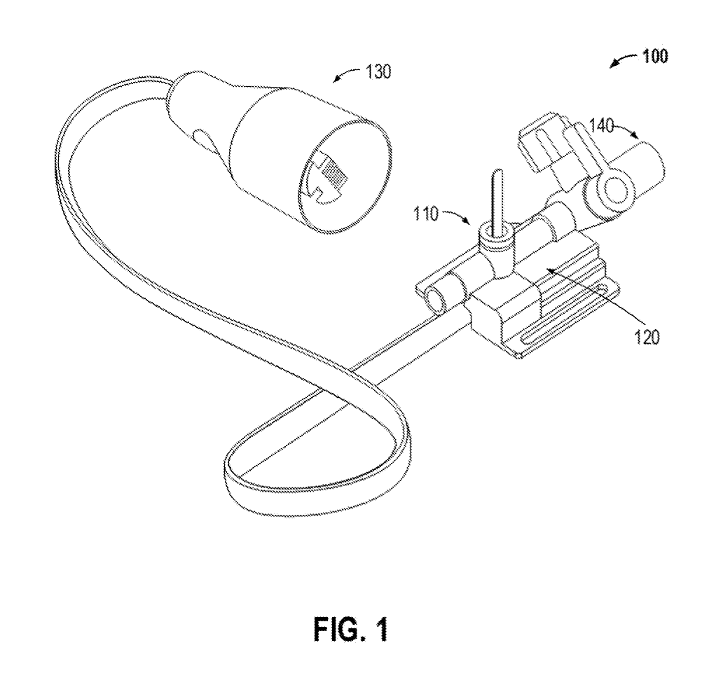

FIG. 1 is a diagram illustrating an example disposable pressure transducer (DPT) 100, with which embodiments of the invention may be practiced. A DPT 100 may include: a plastic enclosure 110 that hosts pressure transducer 120 mounted therewith; a first connector 130 for coupling to a patient monitor; and a second connector 140 that connects to a fluid-filled tubing and catheter, as will be described hereinafter. As can be seen in FIG. 1, the pressure transducer 120 may be located in the plastic enclosure 110 to ensure connectivity to the fluid-filled catheter-tubing system on one side and the patient monitor on the other side.

As described above, to obtain accurate pressure measurements, the DPT 100 must be mounted level with the measurement site. For example, for arterial pressure measurement, the DPT 100 should be mounted at the same level as the patient's heart or the phlebostatic axis. If the DPT 100 is above or below the patient's heart level, a hydrostatic pressure offset occurs due to the height of the fluid column in the tubing, and the apparent pressure signal generated by the DPT 100 is no longer accurately representative of the blood pressure.

The presence of a hydrostatic pressure could introduce significant errors in the pressure measurement. A known method to reduce the effect of the hydrostatic pressure is through manual zeroing of the transducer with respect to atmospheric pressure. The leveling is usually performed by visual estimation or by using a carpenter's level or a laser leveler and includes a number of manual procedures that may be time consuming in many clinical conditions. Moreover, the manual zero referencing is proven to be unreliable with significant inter-user variability. Additionally, the hydrostatic pressure could have a significant impact on the pressure measurement when the patient moves, or when the patient bed is moved or inclined, or when the patient needs to be rotated in a continuous fashion. In these types of clinical conditions, manual zeroing of the pressure transducer could be a challenge.

Embodiments of the invention provide a reliable method for automatic, continuous, and real-time compensation of hydrostatic pressure offset without the need of manual zeroing and leveling. This may provide automatic and real-time compensation for static pressure variations caused by changes in relative height between the measurement site and the DPT mounting location.

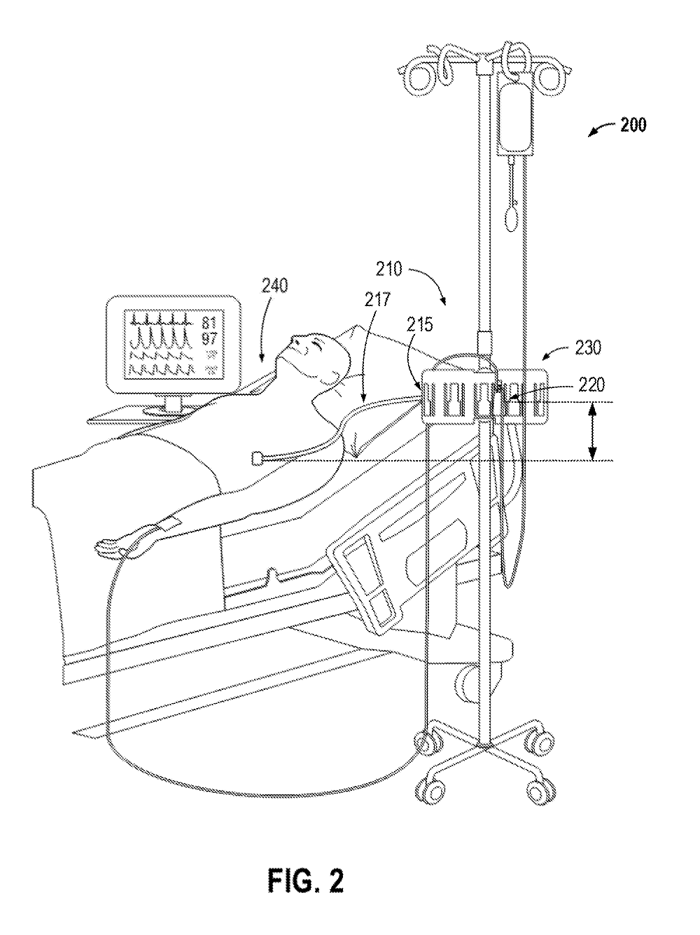

FIG. 2 is a diagram illustrating an example environment 200 in which embodiments of the invention may be practiced. A heart reference system (HRS) 210 including a second pressure transducer 215 (e.g., a reference pressure transducer) separate from the pressure transducer of the DPT 220 used for actual pressure measurement (e.g., a measurement pressure transducer) may be provided to compensate for the hydrostatic pressure (caused by the level difference between the DPT 220 and the measurement site) automatically and in real-time. The pressure transducer 215 of the HRS 210 may be connected to a thin flexible tube 217 filled with blood mimicking fluid, such as water, saline, oil, etc. Therefore, the HRS 210 may include a pressure transducer 215 and a thin flexible tube 217 filled with blood mimicking fluid. One end (shown with a square in FIG. 2) of the tube 217 may be connected at the measurement level such as the patient 240's heart level, while the other end containing the pressure transducer 215 may be connected to a DPT plate 230 or to the DPT 220 directly, such that the second pressure transducer 215 and the measurement pressure transducer of the DPT 220 are at the same level. The pressure transducer 215 in the HRS 210 may measure continuously the pressure difference between the two ends of the tubing, which is proportional to the hydrostatic pressure. The electrical signal from the HRS pressure transducer 215 may then be electronically added to (or subtracted from) the apparent pressure signal from the DPT 220 and the resulting signal will correspond only to the pressure to be measured, such as arterial pressure. In this way the pressure measuring system may operate independently of hydrostatic pressure offsets.

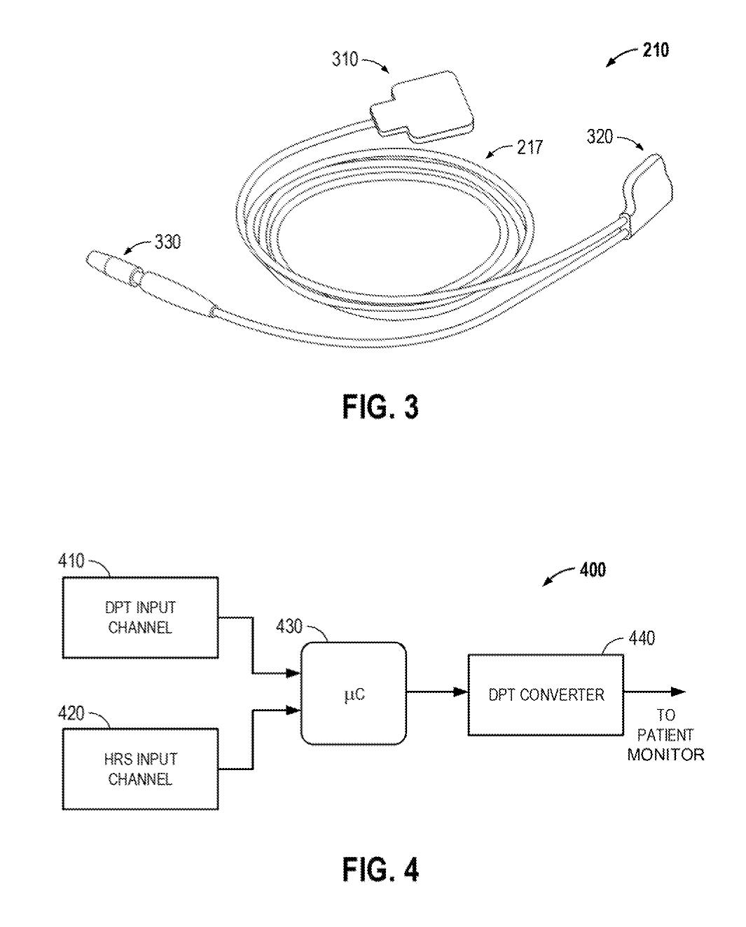

FIG. 3 is a diagram illustrating an example heart reference system (HRS) 210, according to one embodiment of the invention. The HRS 210 may comprise a flexible thin tube 217. Further, as previously described, the tube 217 may be filled with blood mimicking fluid. A first end 310 of the tube 217 may be connected at the measurement level, such as a patient's heart level, while a second end 320 of the tube 217 may contain a pressure sensor (transducer) 215. The pressure transducer 215 may be connected to a DPT plate, or to a DPT directly, as described above, via an electrical connector 330, such that the pressure transducer 215 and the measurement pressure transducer in the DPT are at the same level.

FIG. 4 is a block diagram illustrating an example hydrostatic pressure compensation system 400 comprising electronic modules for reliable, continuous, automatic, and real-time compensation of hydrostatic pressure, according to one embodiment of the invention. DPT input channel 410 and the HRS input channel 420 may be front-end circuits used for signal conditioning of the pressure signals from the DPT and of the HRS. Pressure transducer excitation voltage may be provided and pressure transducer health checks may be performed through the DPT input channel 410 and the HRS input channel 420. The signal conditioning may include using a differential (instrumentation) amplifier to amplify the signal and reduce the common mode voltage. In some embodiments, the pressure signal amplitude may be 5 μV per mmHg per volt of excitation. This signal may be amplified to a predetermined range of volts and the resulting signal may be fed to an analog-to-digital converter (ADC), which could be a part of a microcontroller integrated circuit (μC) 430. The signal conditioning circuits may also include common mode low-pass filters, differential mode low-pass filters, and anti-aliasing filters, etc. Further, the signal conditioning circuits may include some of the transducer health check functions required by the current edition of the IEC (International Electrotechnical Commission) 60601-2-34 standard. The DPT input channel 410 and the HRS input channel 420 operating as front end signal conditioning circuits may also include resistors that pull the open input to an off-scale value for software detection in the event of an open circuit in the pressure signal inputs.

In one embodiment, the firmware of microcontroller 430 may measure the DPT input via the signal conditioning circuits and an internal 24-bit sigma-delta ADC and convert the signal to a pressure value in mmHg. The current positive or negative pressure offset from the HRS channel 420 may be received in the same way and summed with the measured apparent DPT pressure. The compensated/corrected pressure may then be sent to the DPT converter circuit 440. The hydrostatic pressure compensation system 400 may also comprise a power supply module (not shown). In one embodiment described below, power may be harvested from the transducer excitation voltage supplied by the patient monitor.

The DPT converter circuit 440 may convert the digital pressure signal from the microcontroller 430 into a signal that has the exact same characteristics as the pressure signal from the pressure transducer in the DPT, so that the output of the hydrostatic pressure compensation system 400 may be used with patient monitors intended to receive the analog signal from the pressure transducer in the DPT. Therefore, the DPT converter 440 may simulate the ratiometric output of the pressure transducer in the DPT, and may enable a universal connectivity of the hydrostatic pressure compensation system 400 to a wide range of patient monitors, regardless of the type (AC or DC) or the amplitude of the reference (excitation) signal and regardless of the type of the input conditioning or recognition circuitry used in the patient monitors. Because the DPT converter 440 may simulate the analog ratiometric output of the pressure transducer in the DPT, the DPT converter 440 may be alternatively referred to as a DPT simulator.

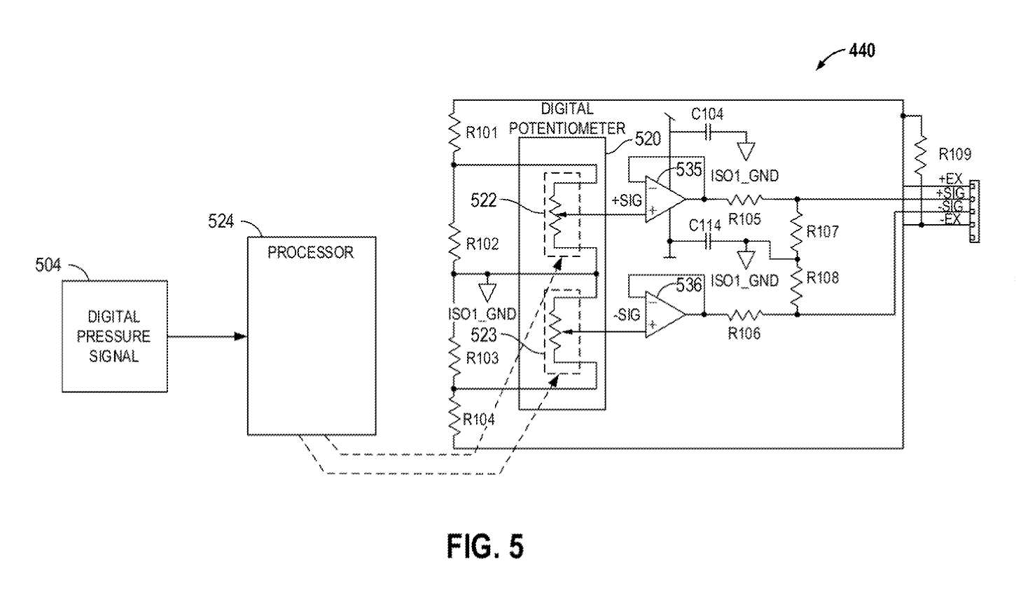

FIG. 5 is a diagram illustrating circuit components of an exemplary DPT converter 440, according to one embodiment of the invention. In this embodiment, the DPT converter 440 may accept the DC or AC excitation voltage (+/−EX) typically applied to industry standard bridge type DPTs and may output a standard differential signal (+/−SIG) of 5 μV per mmHg per volt of excitation. Any excitation voltage within the range of −10 volts to +10 volts at +EX referenced to an excitation reference may be accepted. Moreover, the excitation can be constant or varying.

The DPT converter 440 may include a digital potentiometer 520. The digital potentiometer 520 may be applied in a potentiometer voltage divider mode, analogous to a mechanical potentiometer. The digital potentiometer 520 may perform the same electronic adjustment function as a mechanical potentiometer would with enhanced resolution, solid state reliability, and superior temperature coefficient performance. The wiper position in a digital potentiometer may be commanded digitally instead of by physical movement. In one embodiment, the digital wipers may be commanded through digital signaling by a processor/controller 524 based on the digital DPT pressure signals 504. The controller 524 and the microcontroller 430 of FIG. 4 may be separate, or may be the same controller.

In order to be compatible with a myriad of commercially available patient monitors, the differential output signal between +SIG and −SIG should be ratiometric to the instantaneous applied excitation voltage at 5 μV per mmHg per volt of excitation, and the differential output signal should ride on a common mode level tracking 50% of the instantaneous applied excitation voltage. In addition, commercially available patient monitors may place limits on the allowable zero offset voltage in the zero pressure state, so the circuit should deliver a differential output signal very near zero μV when a zero output is commanded.

Resistor divider R101, R102, R103, and R104 scales the applied excitation voltage down based on the desired full scale differential output voltage. Advantageously, the circuit “ground” reference point may be selected to be at the midpoint of the applied excitation voltage. This would also be the midpoint, or common mode level, of the differential output signal. Therefore, from the circuit perspective, the common mode voltage is zero, or ground, and circuit complexity may thus be reduced.

The pressure to be represented at the signal output may be set by adjusting the wiper positions of the digital potentiometer 520. Because the digital potentiometer 520 may be capable of only limited current and has relatively high and non-constant source impedance, the voltage at wipers 522 and 523 may be unity gain buffered by buffer amplifiers based on operational amplifiers 535 and 536. Those skilled in the art would appreciate that buffer amplifiers may have some offset voltage. An excessive offset voltage could result in an unacceptable zero offset at the differential output, potentially resulting in rejection by some patient monitors. Resistor dividers R105/R107 and R106/R108 may reduce the zero offset of the buffer amplifiers by a factor of 13, ensuring a low zero offset at the differential output.

The R107 and R108 components of the dividers may also provide a 300Ω resistive path between +SIG and −SIG to satisfy a detection requirement of some commercially available patient pressure monitors. Because the desired output is 5 μV per mmHg per volt of excitation with 300 mmHg as full scale, the full scale differential output is 1500 μV per volt of excitation, and the ratio of R101 to R102 may be established taking into account the R105/R107 divider. Those skilled in the art would recognize that other divider ratios could be used at R101 and R102 to accommodate other full scale ranges, or other ratios at R105/R107.

Current consensus standards for patient pressure monitors may require the monitor to perform continuous health checks on DPT devices, and promptly signal a technical fault for a bad transducer. In view of the fact that older monitors do not generally support such health checks and may be limited to detection of a transducer being connected or disconnected, a combined health check and transducer detection function may be implemented within the DPT converter circuit 440, which may simulate a transducer-connected signal to be outputted to the patient monitor when a connected DPT passes the health check, and a transducer-disconnected signal when no DPT is connected or when a connected DPT fails the health check.

In one embodiment, the hydrostatic pressure compensation system 400 illustrated in FIG. 4 may also include a power supply module that harvests electrical power from the excitation pin of the pressure transducer connector of the patient monitor to power the pressure compensation system 400 and one or more DPTs and the HRS. In one embodiment, the power supply module may include a transformer, which may provide galvanic isolation between the excitation voltage supplied by the patient monitor and the power supplied to the pressure compensation system 400, and between the power to the common instrument circuitry and to the patient interface circuits.

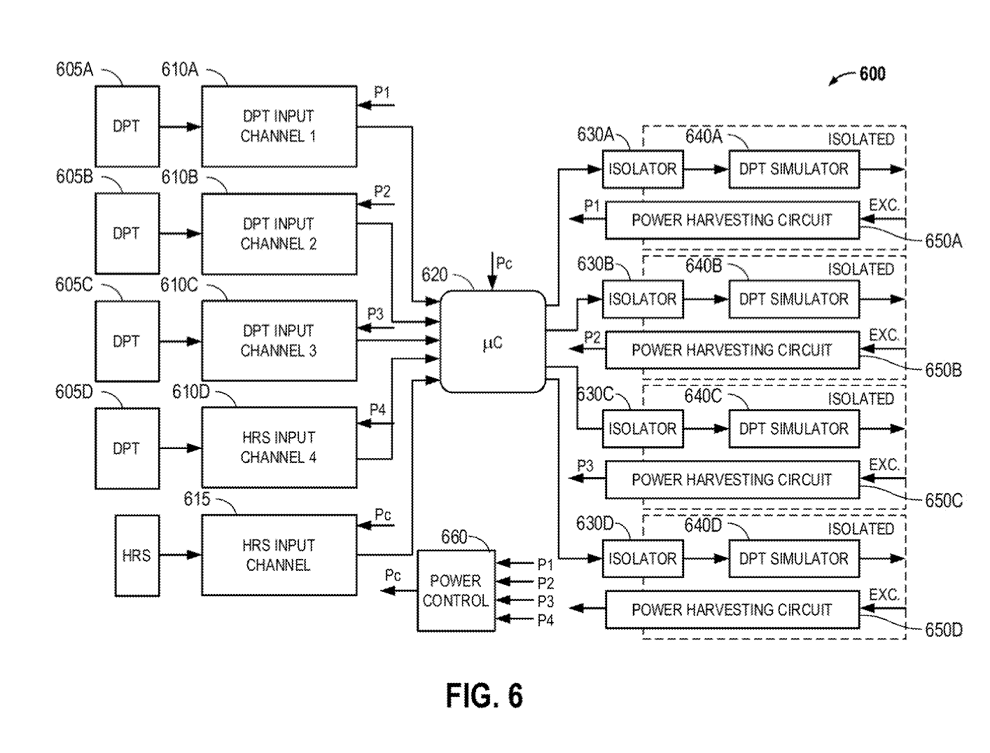

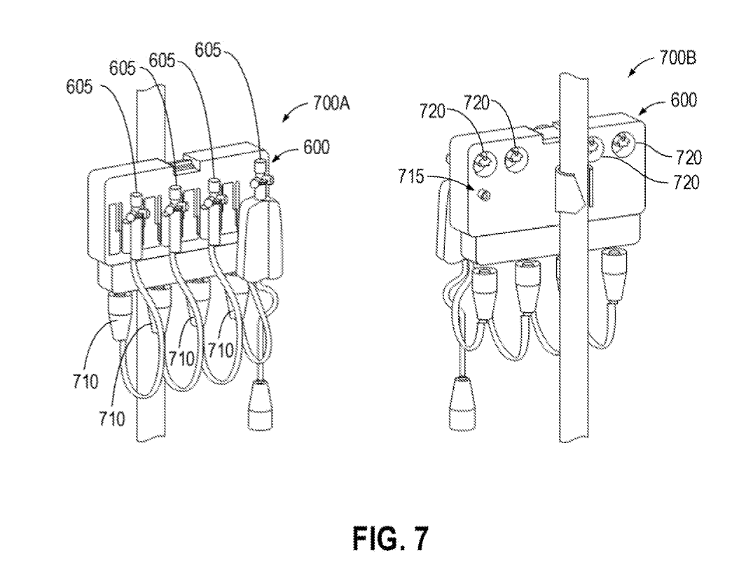

Referring to FIGS. 6 and 7, an example DPT plate 600 including a hydrostatic pressure compensation system that can hold up to four separate DPTs 605A-D is shown. FIG. 6 is a diagram illustrating the exemplary DPT plate 600 comprising various electronic modules. Four individual DPT input channels 610A-D, each being able to connect a separate DPT 605, may be provided and connected to a microcontroller 620. An HRS input channel 615 may be provided and connected to the microcontroller 620 to receive the HRS output for hydrostatic pressure compensation. Thus, the microcontroller 620 may receive four separate DPT inputs and an HRS input, convert these inputs into digital signals representative of the respective pressure values, correct digital DPT signals by compensating for the hydrostatic pressure based on the digital HRS pressure signal, and output the corrected digital DPT signals associated with each of the four DPT channels. Four isolators 630A-D and four DPT simulators 640A-D may be provided. Corrected digital DPT pressure signals associated with each of the DPT channels may be inputted into a respective DPT simulator 640 through a respective isolator 630. In each DPT simulator 640 an analog signal that has the exact same characteristics as the pressure signal from the pressure transducer in the DPT is generated based on the corrected digital DPT pressure signals and outputted to a patient monitor supporting four DPT channels. Power harvesting circuits 650A-D may harvest the power supplied as the excitation voltage by the patient monitor to power the whole system. Each power harvesting circuit 650 may supply power to the respective DPT input channel 610 and the DPT 605 connected to the channel. A power control module 660 may combine the outputs of the power harvesting circuits 650A-D and provide power to the HRS input channel 615 and the microcontroller 620.

FIG. 7 illustrates a front view 700A and a back view 700B of the example DPT plate 600. Four DPTs 605 are illustrated removably attached to the front side of the DPT plate 600 and connected to four DPT input connectors 710. Each DPT input connector 710 may corresponds to one DPT input channel 610 illustrated in FIG. 6. On the back side of the DPT plate 600, one HRS input connector 715 and four DPT output connectors 720 may be provided. The HRS input connector 715 may correspond to the HRS input channel 615 of FIG. 6. The DPT output connectors 720 may be connected to the DPT channels of a patient monitor, as described above.

Therefore, embodiments of the invention provide a method for reliably, automatically, and continuously compensating in real-time for hydrostatic pressure in DPT measurements without manual leveling or zeroing. An HRS including a transducer and a fluid-filled flexible thin tube may be provided to measure the hydrostatic pressure, which may be used in the correction of apparent transducer measurements of venous, arterial, or intracranial pressures, etc. A DPT simulator (converter) that can simulate the analog ratiometric output of a DPT based on the equivalent digital pressure signals using a digital potentiometer has been described. Other circuitries, such as the combined DPT connection/health check detection circuit and the power harvesting circuit, have been described and may be used in different embodiments of the invention.

It should be appreciated that aspects of the invention previously described may be implemented in conjunction with the execution of instructions by controllers, such as (micro) controller 430, 524, or 620. Controller 430, 524, or 620 may operate under the control of a program, routine, or the execution of instructions to execute methods or processes in accordance with embodiments of the invention. For example, such a program may be implemented in firmware or software (e.g. stored in memory and/or other locations) and may be implemented by controllers and/or other circuitry. Further, it should be appreciated that the terms processor, microprocessor, circuitry, controller, microcontroller, etc., refer to any type of logic or circuitry capable of executing logic, commands, instructions, software, firmware, functionality, etc.

The various illustrative logical blocks, modules, and circuits described in connection with the embodiments disclosed herein may be implemented or performed with a general purpose processor, a microcontroller, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor may be a microprocessor or any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A system for compensating for hydrostatic pressure offset in transducer-based pressure measurements, comprising:
a measurement pressure transducer to measure an apparent fluid pressure at a measurement site;
a reference pressure transducer to measure a hydrostatic pressure caused by a level difference between the measurement pressure transducer and the measurement site; and
a controller to generate a corrected fluid pressure measurement based on the apparent fluid pressure and the hydrostatic pressure, wherein the measurement pressure transducer and the reference pressure transducer are placed at a same first level, and the measurement site and an end of a fluid-filled tube connected to the reference pressure transducer are at a same second level.

2. The system of claim 1, wherein the measurement site is one of a vein, artery, or cranium, and the fluid-filled tube connected to the reference pressure transducer is filled with a blood mimicking fluid.

3. The system of claim 1, wherein an output of the measurement pressure transducer and an output of the reference pressure transducer are amplified and converted into digital signals, and the controller generates the corrected fluid pressure by summing the converted output of the reference pressure transducer and the converted output of the measurement pressure transducer.

4. The system of claim 3, further comprising a pressure transducer simulator comprising a digital potentiometer to simulate a pressure transducer outputting an analog ratiometric signal representative of the corrected fluid pressure.

5. The system of claim 4, further comprising a hardware module that generates a pressure transducer connected/disconnected signal based on whether the measurement pressure transducer is connected and passes a health check.

6. The system of claim 3, further comprising a power harvesting circuit to harvest power from a transducer excitation voltage pin of a patient monitor to power the measurement pressure transducer, the reference pressure transducer, and the controller.

7. The system of claim 6, wherein the power harvesting circuit includes a transformer to provide galvanic isolation.

8. A method for compensating for hydrostatic pressure offset in transducer-based pressure measurements, comprising:
measuring an apparent fluid pressure at a measurement site with a measurement pressure transducer;
measuring a hydrostatic pressure caused by a level difference between the measurement pressure transducer and the measurement site with a reference pressure transducer; and
generating a corrected fluid pressure measurement based on the apparent fluid pressure and the hydrostatic pressure with a controller, wherein the measurement pressure transducer and the reference pressure transducer are placed at a same first level, and the measurement site and an end of a fluid-filled tube connected to the reference pressure transducer are at a same second level.

9. The method of claim 8, wherein the measurement site is one of a vein, artery, or cranium, and the fluid-filled tube connected to the reference pressure transducer is filled with a blood mimicking fluid.

10. The method of claim 8, wherein an output of the measurement pressure transducer and an output of the reference pressure transducer are amplified and converted into digital signals, and the controller generates the corrected fluid pressure by summing the converted output of the reference pressure transducer and the converted output of the measurement pressure transducer.

11. The method of claim 10, further comprising simulating a pressure transducer outputting an analog ratiometric signal representative of the corrected fluid pressure with a pressure transducer simulator comprising a digital potentiometer.

12. The method of claim 11, further comprising generating a pressure transducer connected/disconnected signal based on whether the measurement pressure transducer is connected and passes a health check.

13. The method of claim 10, harvesting power from a transducer excitation voltage pin of a patient monitor with a power harvesting circuit to power the measurement pressure transducer, the reference pressure transducer, and the controller.

14. The method of claim 13, wherein the power harvesting circuit includes a transformer to provide galvanic isolation.
